(19) United States
(12) Patent Application Publication
(10) Pub. No.: US 2016/0001082 A1
(43) Pub. Date: Jan. 7, 2016

(54) SELECTIVE BLOCK OF NERVE ACTION POTENTIAL CONDUCTION

(71) Applicant: Georgia Tech Research Corporation, Atlanta, GA (US)

(72) Inventors: Robert Butera, Decatur, GA (US); Yogi Anil Patel, Atlanta, GA (US); Laveeta Joseph, Cincinnati, OH (US)

(21) Appl. No.: 14/790,474

(22) Filed: Jul. 2, 2015

Related U.S. Application Data
(60) Provisional application No. 62/020,430, filed on Jul. 3, 2014.

(51) Int. Cl. A61N 1/36; A61N 1/05

(52) U.S. Cl. CPC A61N 1/36171; A61N 1/0556; A61N 1/36053; A61N 1/36057; A61N 1/36067; A61N 1/36071

(57) ABSTRACT

A method of selectively blocking a portion of a nerve signal is disclosed. The method may include the step of providing an electrode around a subject's peripheral nerve and connecting the electrode to a stimulator. The stimulator may then energize the electrode with a continuous periodic waveform of at least 50 kHz. This energization of the electrode can result in the selective block of one of: 1) a fast portion of a nerve signal having a conduction velocity greater than 2 m/s, and 2) a slow portion of the nerve signal having a conduction velocity less than 2 m/s. A method according to the present disclosure may allow the non-blocked portion of the nerve signal to be conducted substantially unimpeded.

### CROSS-REFERENCE TO RELATED APPLICATIONS

This application claims the benefit of U.S. Provisional Application No. 62/020,430, titled “Selective Block of Sensory or Motor Nerve Action potential Conduction via Electrical Stimulation,” filed 3 Jul. 2014, and which is fully incorporated by reference.

### FIELD OF THE INVENTION

The present invention is directed to a method for selectively blocking nerve conduction, and more particularly to a method of blocking either sensory or afferent signaling by the use of kilohertz high frequency alternating current.

### DESCRIPTION OF THE RELATED ART

Electrical stimulation for the modulation of peripheral nerve activity is utilized for the treatment of neuropathological diseases. In some cases, it is desirable to selectively inhibit specific fibers within a peripheral nerve. Various thermal, mechanical, and pharmacological methods have been used for selective blocking but are either slow acting, or not quickly reversible, rendering them unsuitable for chronic applications.

Electrical stimulation with kilohertz high-frequency alternating current (KHFAC) has been shown to be effective at blocking action potential conduction in peripheral nerves. The method is quick, reversible, and currently employed in a variety of clinical applications, including appetite control, bladder control, and post-amputation pain relief.

The use of KHFAC to modify nerve conduction and activity was first published in 1962 by Tanner. Since then, very few labs around the world have used this technique or actively investigate it. Most commonly, work in this area has focused on the use of KHFAC to inhibit motor activity for applications in neural prosthetics. Other studies have also contributed to the field through investigations the use of KHFAC on a specific organ system, such as the bladder, or to block post-amputation pain.

Studies on KHFAC have consistently shown that it is fully reversible and repeatable. KHFAC has received increased attention from investigators for applications in functional electrical stimulation and neuromodulation.

Prior work on KHFAC has been conducted on a variety of animal species, such as sea slugs and frogs. Through the use of suction electrodes and frequencies ranging from 1-50 kHz, this work demonstrated that varying the frequencies had a direct impact on the block threshold, and that the impact was different with nerve fibers of differing conductions and complexities. Unlike previous implementations, this block showed indications of being fiber specific; however suction electrodes are impractical for in vivo or chronic applications.

The ability to selectively block nerve conduction in more complex mammalian peripheral nerves, and particularly the ability to selectively block motor or pain signals in vivo, has great utility in the treatment of many clinical conditions. While this early work intimated at a possible clinical application of KHFAC nerve block, many aspects of the research, including the use of suction electrodes, left a great deal to be resolved before KHFAC block could find practical applications.

Thus, a need exists for a method of selectively blocking nerve conduction in different types of mammalian nerve fibers, such as those associated with sensation or motor signals.

### BRIEF SUMMARY OF THE INVENTION

The present invention comprises a method of selectively blocking a portion of a nerve signal. Embodiments of the present disclosure may include the step of providing an electrode around a subject's peripheral nerve and connecting the electrode to a stimulator.

The stimulator may then energize the electrode with a continuous periodic waveform of at least 50 kHz. This energization of the electrode can result in the selective block of one of: 1) a fast portion of a nerve signal having a conduction velocity greater than 2 m/s, and 2) a slow portion of the nerve signal having a conduction velocity less than 2 m/s. A method according to the present disclosure may allow the non-blocked portion of the nerve signal to be conducted substantially unimpeded.

In some embodiments of the present disclosure, the periodic waveform provided by the stimulator is about 70 kHz. The electrode may be of a tripolar cuff-type. For example and not limitation, embodiments in accordance with the present disclosure may involve the electrode being applied in the vicinity of a sciatic nerve or a vagus nerve.

In some embodiments of the present disclosure, the selective nerve block may be verified by measuring for a muscular contraction. In addition or in the alternative, the selective nerve block may be verified by measuring for a nerve signal propagation using a second electrode.

In accordance with the present disclosure, the periodic waveform provided by the stimulator may be adjusted such that a desired indication of the selective nerve block is observed. This adjustment may be made while monitoring indicia of nerve block in real time while the stimulator is adjusted. Alternatively or additionally, the periodic waveform provided by the stimulator can be adjusted such that a desired indication of the conduction of the non-blocked portion of the nerve signal is observed.

Other aspects and features of embodiments of the present invention will become apparent to those of ordinary skill in the art, upon reviewing the following detailed description in conjunction with the accompanying figures.

### BRIEF DESCRIPTION OF THE FIGURES

The various embodiments of the disclosure can be better understood with reference to the following drawings. The components in the drawings are not necessarily to scale, emphasis instead being placed upon clearly illustrating the principles of the various embodiments of the present invention. In the drawings, like reference numerals designate corresponding parts throughout the several views.

FIG. 1A illustrates a setup for producing KHFAC in a rat sciatic nerve.

FIG. 1B illustrates a schematic of nerve cuff electrodes.

FIG. 2A illustrates a circuit diagram of a setup for measuring the attenuated output.

FIG. 2B illustrates a plot of current against frequency for inputs of 1V (grey) and 2V (black).

FIG. 3A illustrates the selective KHFAC block of electrically evoked fast and slow CAP components in rat sciatic nerve.

FIG. 3B illustrates the selective KHFAC block of electrically evoked fast and slow CAP components in rat vagus nerve.

FIG. 3C illustrates an example recording of the onset response as recorded by electrode V2 when KHFAC (20 kHz at 1 mA) is delivered to the nerve with a bandpass (100 Hz-10 kHz) filter.

FIG. 3D illustrates a plot of the onset response duration (mean and standard deviation, n=3) for select KHFAC frequencies at 1 mA.

FIG. 4 illustrates the individual measured data of the KHFAC conduction block in the sciatic rat nerve.

FIG. 5 illustrates a plot of amplitude against frequency for the fast and slow components for electrically evoked motor output of the sciatic nerve.

FIG. 6 illustrates a plot of amplitude against frequency for the fast and slow components of the sciatic nerve.

FIG. 7 illustrates a plot of amplitude against frequency for the fast and slow components of the vagus nerve.

### DETAILED DESCRIPTION OF PREFERRED EMBODIMENTS

Although preferred embodiments of the invention are explained in detail, it is to be understood that other embodiments are contemplated. Accordingly, it is not intended that the invention is limited in its scope to the details of construction and arrangement of components set forth in the following description or illustrated in the drawings. The invention is capable of other embodiments and of being practiced or carried out in various ways. Also, in describing the preferred embodiments, specific terminology will be resorted to for the sake of clarity.

It must also be noted that, as used in the specification and the appended claims, the singular forms “a,” “an” and “the” include plural referents unless the context clearly dictates otherwise.

Also, in describing the preferred embodiments, terminology will be resorted to for the sake of clarity. It is intended that each term contemplates its broadest meaning as understood by those skilled in the art and includes all technical equivalents which operate in a similar manner to accomplish a similar purpose.

Ranges may be expressed herein as from “about” or “approximately” one particular value and/or to “about” or “approximately” another particular value. When such a range is expressed, another embodiment includes from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

As used in the specification and claims, a biomarker can be any biological entity (protein, enzyme, hormone, catecholamine, etc.) that is released from an organ or others to have some physiological effect (increased heart rate, increased respiration, sweating, etc.). As used in the specification and claims, somatic nerves are defined as those primarily related to motor and sensory control, and including but not limited to the sciatic nerve, the median nerve, and the ulnar nerve. As used in the specification and claims, autonomic nerves are defined as any nerve involved with controlling function of automatic functions, including but not limited to, breathing, heart rate, blood sugar levels, and hunger. Examples of these nerves include but are not limited to, the phrenic nerve, the hypoglossal nerve, and the cervical vagus nerve and its branches, including but not limited to the hepatic branch, the cardiac branch, the gastrointestinal branch. As used in the specification and claims, the term sympathetic trunk is defined as a chain of neurons and axons that control numerous autonomic and somatic activities, including but not limited to, monitoring and controlling the functioning of organs such as the heart, the kidneys, and/or the liver.

It is also to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

Experimental Preparation

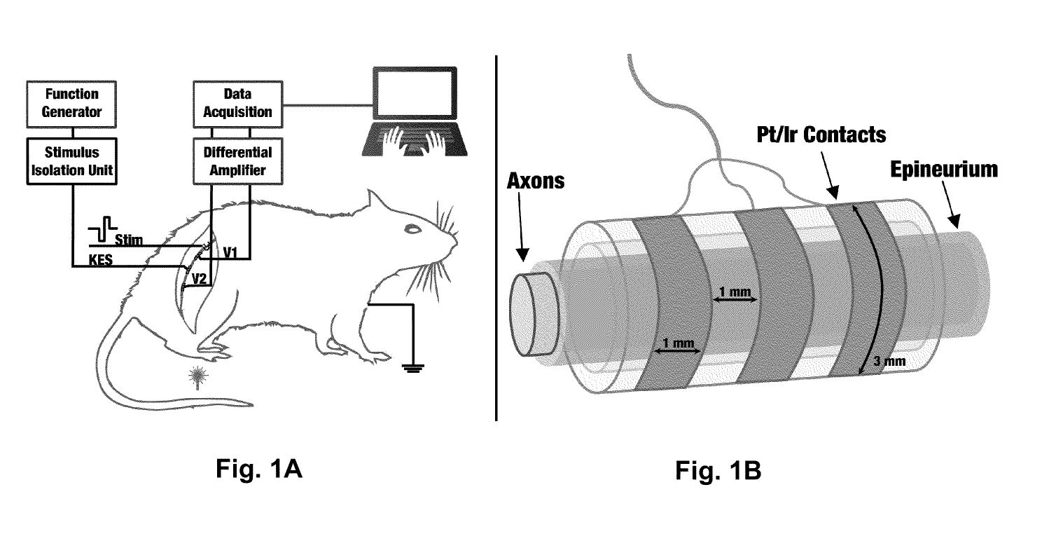

In accordance with the present disclosure, in vivo acute experiments were performed on the left and right sciatic nerves of four Lewis rats as well as the vagus nerves of four additional Lewis rats. Rats were anesthetized with 5% isoflurane and fixed in the prone position prior to surgery. Anesthesia was maintained at 2-3% for the first 45 minutes and 1-1.5% for the remainder of the experiment using a nose cone. Ophthalmic ointment was applied to both eyes to prevent drying. The animal's toe pinch reflex was used to maintain surgical anesthetic depth throughout the experiment. Body temperature and circulation were maintained via a heating pad at 37° C. The right thigh of the animal was shaved and the biceps femoris muscle was separated for sciatic nerve preparations. The sciatic nerve was exposed from the top of the biceps femoris to the bottom of the gastrocnemius muscle in the ankle and cuff electrodes were placed around the nerve (FIG. 1A). A total of 3-4 cm was exposed in all sciatic nerve preparations. Sterile rat ringer solution was applied throughout the experiment to prevent muscle and nerve tissue dehydration. After completing the experimental protocol on the animal's right side, the wound site was closed with surgical clips. The same procedure was executed on the animal's left sciatic nerve. The same sterile procedures were used to expose and experiment on the left cervical vagus nerve. Preparations lasted an average of 5 hours after which animals were euthanized via carbon dioxide. All experiments were conducted at room temperature.

Electrophysiological Configuration and Measurement

Recordings of CAP propagation along the nerve were used as an output measure to detect and monitor the status of selective conduction block in both sciatic and vagus nerves. A combination of hook and tripolar cuff electrodes were used to conduct these studies. A bipolar stainless steel hook electrode was used to electrically elicit CAPs. Tripolar cuff electrodes (FIG. 1B) were used to record CAPs and deliver the KHFAC block stimulus. The sampling rate of the data acquisition system (Digidata 1322, Molecular Devices, Foster City, Calif., full scale range ±10.096V) was 50 kHz per channel. Nerve recordings were differentially amplified (Model 1700, A-M Systems, Sequim, Wash.) with a gain of 1000×, and filtered using a second-order bandpass (100 Hz-5 kHz) filter. In some cases, an additional digital first-order bandpass (300 Hz-3 kHz) filter was enabled in the acquisition system to enhance visualization of the CAP during application of the KHFAC stimuli. This experimental setup provided direct monitoring of the neural activity along the nerve and the status of selective KHFAC conduction block. FIG. 1 displays the experimental setup used for sciatic nerve studies. No significant modifications were made to this setup for the vagus nerve studies. Vagus nerve studies positioned the electrode on the cervical section of the vagus nerve.

Electrode Design and Fabrication

Nerve recordings and application of the KHFAC block stimulus were performed using custom-made, tripolar, longitudinally slit cuff electrodes as shown in FIG. 1B. Cuff electrodes were made using silicone tubing, stainless steel wire, and platinum-iridium (Pt—Ir, 90/10) contacts (3 mm×1 mm). Average cuff diameter and length for sciatic nerve studies were 1.25-1.5 mm and 5 mm, respectively, with 1 mm between Pt—Ir contacts. Average cuff diameter and length for vagus nerve studies were 1.0-1.2 mm and 3 mm, respectively, with 0.75 mm between Pt—Ir contacts. Impedance was characterized for all cuffs using an impedance conditioning module (FHC, Bowdoin, Me.). The impedance range for the recording and KHFAC stimuli cuffs were 1.6-2.0 kΩ and 1-1.2 kΩ, respectively at 1 kHz.

Stimulation

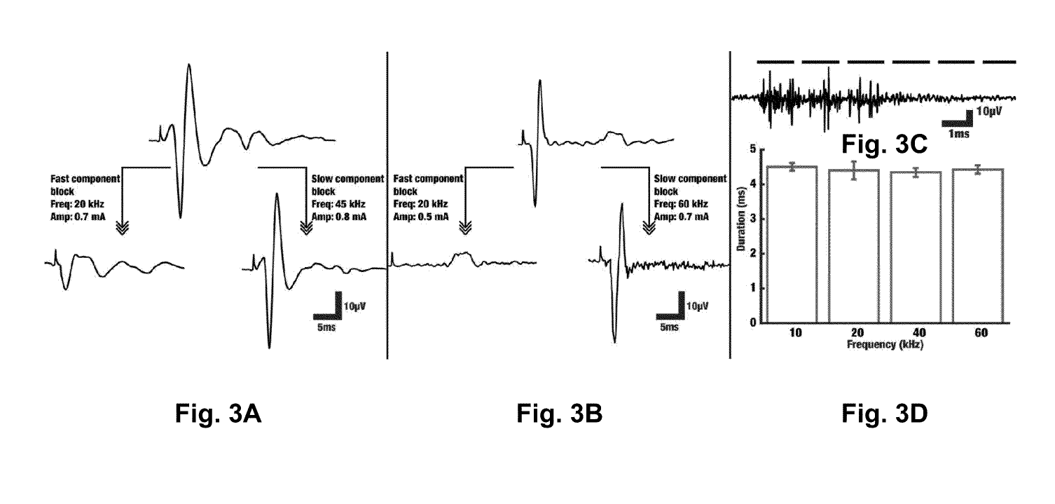

Electrical stimulation was used to elicit CAPs in both sciatic and vagus nerves. Supra-maximal cathode-first biphasic electrical stimulus pulses (5 V, 0.2 ms) were generated by the data acquisition system to trigger CAPs in the nerve. Sensory stimulation in the form of a hot air gun was used to deliver a low flow, high heat noxious stimulus to the forepaw of the animal. While the KHFAC block waveform may be generated by a voltage source or a current source, in this case the block waveform was generated using a function generator (DS345, Stanford Research Systems, Sunnyvale, Calif.). Both the stimulation pulses and KHFAC block waveform were converted to current sources (1 mA/V) by optically-isolated stimulus isolation units (Model 2200, A-M Systems, Sequim, Wash.). All stimulation equipment was calibrated and offsets were zeroed prior to experimentation to ensure no leakage of current from equipment. Evoked CAPs have two visually distinguishable components that are referred to as the fast and slow responses, in reference to their time of appearance relative to the stimulus artifact that occurs when a nerve is electrically stimulated. The fast response is attributed to large diameter, predominantly (but not exclusively) myelinated fibers with fast conduction velocities (e.g., Aα, Aβ, Aγ) and the slow response is attributed to small diameter, predominantly (but not exclusively) unmyelinated fibers with slow conduction velocities (e.g., C) (Gasser 1941). Components with conduction velocities below 2 m/s were classified as slow and components with conduction velocities greater than 2 m/s were classified as fast. In addition, the waveforms associated with these two components were visually recognizable (FIG. 3A-B). During experimentation, fast and slow components were identified by latencies between stimulus onset and the recording electrodes. Fast and slow components were associated with latencies in the range of 0.5-1.5 ms and 9-13 ms for electrode V1, respectively. In addition, fast and slow components were associated with latencies in the range of 3-5 ms and 15-20 ms for electrode V2, respectively.

Force Transduction

Force transduction experiments were carried out to validate the functionality of selective block of the fast component. The posterior segment of the leg was shaved and the biceps femoris was exposed to allow dissection of the gastrocnemius-soleus muscle complex. The tibia was fixed to the experimental rig and the Achilles tendon was attached to a force transducer (Model 724490, Harvard Apparatus) using a hemostat.

KHFAC Conduction Block Trials

Block (or no block) of the slow component was verified via visual classification. First, block was visually verified in-line during experimentation. Block did not occur when there was a repeatedly triggered and identifiable waveform in the latency window of the slow component. Block occurred when there was no identifiable and repeatable waveform in this latency window. This visual analysis was repeated during post-hoc data analysis. Furthermore, the change in rectified and integrated area of both the fast and slow components of the CAP was quantified. Fast and slow components were identified using measured latencies and conduction velocities. The identified components were numerically integrated using Romberg's method for numerical integration. Romberg's method was chosen because of its simplicity and ability to eliminate error without the need for oversampling. Because of these qualities, Romberg's method is more suitable for integrating experimental data, which tends to be noisy.

Attenuation of Stimulus Isolation Units at High Frequencies

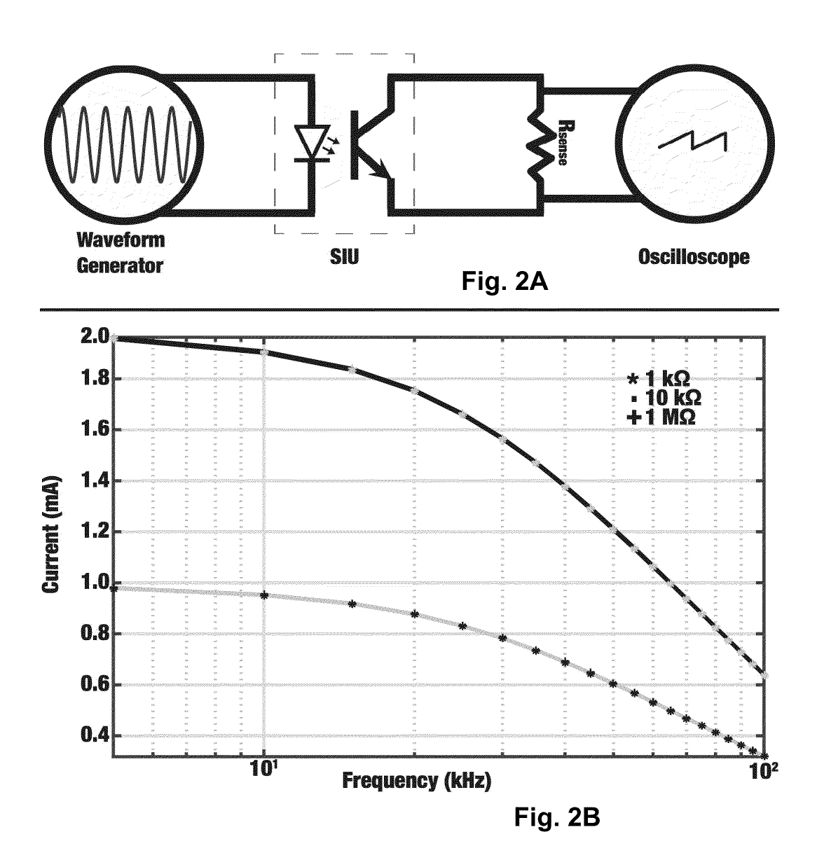

Frequencies up to 50 kHz and 70 kHz were tested for the sciatic and vagus nerve experiments, respectively. These frequency ranges are beyond the rating (up to 40 kHz) of the stimulus isolation units (SIUs) driven by the waveform generator. The SIUs can provide outputs at frequencies higher than the ratings, however the output is of a lower amplitude than specified. This attenuation was measured and used this information to calculate the true current output at a given frequency. Attenuation of the KHFAC waveform was characterized by measuring the current across varying resistances (1 kΩ, 10 kΩ, and 1 MΩ) in parallel with the output terminal of the SIU, as shown in FIG. 2A. The resulting current across the resistor was calculated using voltage measurements made using an oscilloscope (HP 54602B, Hewlett Packard, Palo Alto, Calif.). The resulting calibration curves (FIG. 2B) were used to calculate the true current provided by the KHFAC. These adjusted values are reported in this manuscript. The same trends qualitatively reported are also evident in the non-adjusted data.

Results

Each experimental preparation was tested for normal conduction properties prior to beginning selective KHFAC trials. CAPs were triggered using supra-threshold electrical or sensory stimulation. Evoked nerve activity was recorded using cuff electrodes along the length of the nerve. The distance between stimulating and recording electrodes was used to determine conduction velocities for the CAP components.

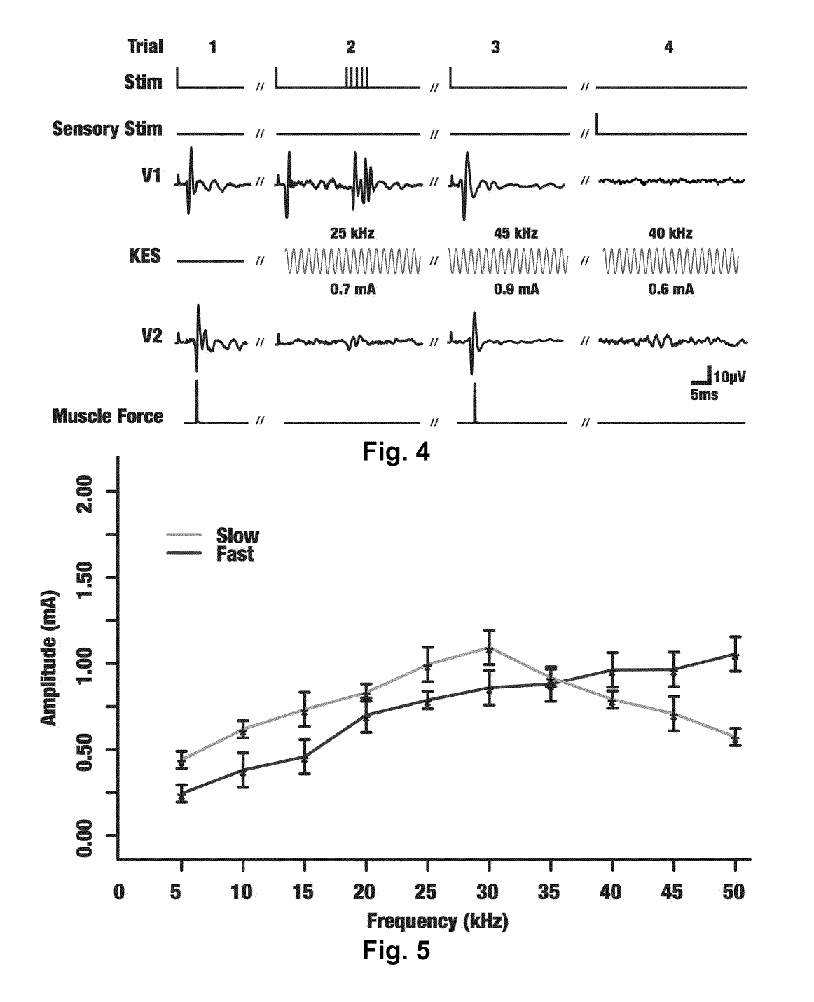

FIG. 4 shows single trial data from one experiment demonstrating selective KHFAC block. The labels on the left correspond to the electrodes shown in FIG. 1. Stimulus artifacts precede the fast component and occur earlier than the time traces shown in V1 and V2. Trial #1 shows the standard baseline trial conducted throughout every experiment to verify CAP initiation and propagation. A supra-threshold stimulation pulse is delivered to the nerve at the proximal end and the propagating CAP and associated muscle twitch are recorded. Trial #2 depicts the case where a CAP is triggered and the fast component is selectively blocked via KHFAC. This block of the fast component led to an absence of the muscle twitch but maintained propagation of the slow component. Additional stimulation pulses were delivered to the nerve during selective block of the fast component to ensure true block had occurred. The additional stimulation pulses evoked the fast component response in the proximal recording electrode but not in the distal recording electrode, demonstrating the continued effects of the KHFAC selective block. This resulted in continued absence of muscle force generation while maintaining propagation of the slow component. Trial #3 demonstrates a scenario where the slow component is selectively blocked, leaving the fast component and muscle force intact. Trial #4 shows the use of sensory stimulation (heat) to evoke the slow component only. The stimulus is applied to the hind leg of the animal, resulting in the CAP appearing on electrode V2 first and then being blocked (absent on V1). Individual trials, as shown in FIG. 4, are aggregated to produce block threshold characterization curves (FIGS. 5-7).

The primary results from these studies are the block threshold characterization curves (FIGS. 5-7). These curves provide a visual representation of the frequency and amplitude pairs that allow for selective KHFAC block of CAP components. FIG. 5 depicts that KHFAC amplitudes greater than or equal to the dark grey line but less than the light grey line provide for selective block of the fast component for frequencies up to 35 kHz. Similarly, KHFAC amplitudes greater than or equal to the light grey line but less than the dark grey line provide for selective block of the slow component for frequencies between 35-50 kHz. This interpretation applies to all the block threshold characterization curves presented here.

CAP Components Corresponding to Sensory Stimuli and Motor Output are Selectively Blocked

Selective KHFAC block was utilized to demonstrate loss of either motor function or sensory evoked CAPs when either the fast or slow component was selectively blocked. Sensory stimulation was applied to the hind leg of the animal to evoke sensory CAPs and evaluate block of the slow component. Electrical stimulation was used to evoke motor output and evaluate block of the fast component. Motor block was verified by force measurements (FIG. 4, Trial #2) and sensory CAP generation and block was verified by direct nerve recordings (FIG. 4, Trial #4). The block threshold was characterized for both sensory evoked CAPs and motor output (FIG. 5). The fast component block threshold increased with frequency while the slow component block threshold displayed a non-monotonic trend peaking around 30 kHz.

Fast and Slow Components of the Triggered CAP are Selectively Blocked by KHFAC

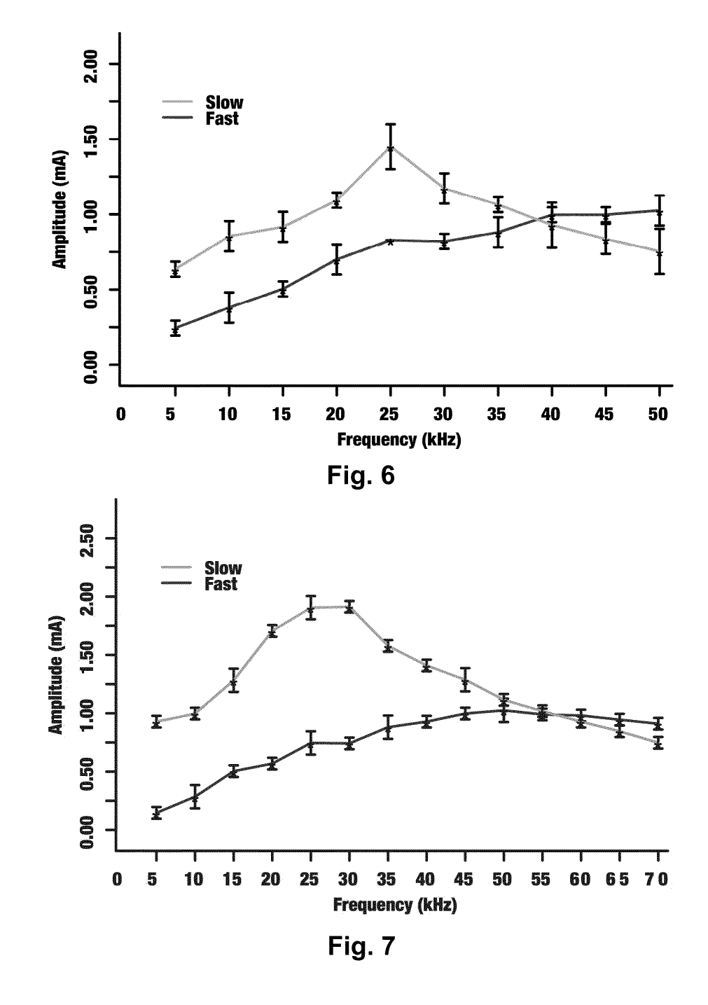

Selective KHFAC block was achieved of the fast (FIG. 4, Trial #2) and slow (FIG. 4, Trial #3) components of electrically triggered CAPs in 8 rat sciatic nerves. FIG. 6 shows the block threshold characterization for KHFAC stimuli up to 50 kHz. The non-monotonic block threshold trend peaked around 25 kHz.

Selective Block of CAP Components can be Achieved in Multiple Nerve Types

The robustness of selective KHFAC conduction block was also examined in the rat vagus nerve preparation. The experimental setup (FIG. 1) was modified by using smaller cuff electrodes for interfacing with the rat vagus nerve. Electrical stimulation was used to evoke CAPs and nerve recordings were used to assess status of block (FIG. 3B). FIG. 7 shows the mean and standard deviation for selective KHFAC block thresholds in the rat vagus nerve. The trends are similar to the sciatic nerve results in FIG. 6. The fast component block threshold increases monotonically while the slow component block threshold displays a non-monotonic trend peaking around 30 kHz.

Changes in Rectified and Integrated Area of CAP Components

The resolution of selective KHFAC conduction block was quantified in the rat sciatic nerve by rectifying and integrating the fast and slow components.

TABLE 1

| Frequency (kHz) | Amplitude (mA) | Fast | Slow |
|---|---|---|---|
| 0 | 0.0 | 0.0 ± 0.0 | 0.0 ± 0.0 |
| 20 | 0.5 | 13.6 ± 1.5 | 2.0 ± 0.3 |
| 20 | 1.0 | 99.0 ± 0.5 | 12.3 ± 0.8 |
| 20 | 1.5 | 99.0 ± 0.5 | 99.0 ± 0.5 |
| 40 | 0.5 | 8.7 ± 2.4 | 24.1 ± 3.6 |
| q 40 | 0.9 | 18.2 ± 5.3 | 99.0 ± 0.5 |
| 40 | 1.5 | 99.0 ± 0.5 | 99.0 ± 0.5 |

Table 1 depicts example data from two arbitrarily chosen frequencies and increasing amplitudes. The fast and slow components were rectified and integrated to quantify the percent reduction in the rectified and integrated area of each component compared to baseline (0% reduction) as a function of the block frequency and amplitude. It can be seen that the rectified and integrated area of the fast and slow components of the CAP decrease in a frequency and amplitude dependent manner. For example, the fast component area decreases significantly as the KHFAC amplitude is increased at low frequencies (20 kHz). Simultaneously, the slow component demonstrates a decrease, until both are no longer identifiable by our classification method. The same trend is observed at high frequencies (40 kHz), in which the slow component rectified and integrated area significantly decreases with increasing amplitude.

Discussion

The present disclosure is the first to experimentally explore the frequency-amplitude relationship of the different components of the CAP of a mixed composition mammalian nerve to KHFAC stimulation. The results show that KHFAC stimulation can induce selective conduction block in whole nerves. Further, the present disclosure demonstrates the differential response of fast and slow mammalian fibers to KHFAC stimulation, and demonstrates that the fast and slow components of CAPs can consistently and selectively be blocked in multiple different mammalian nerves. This was validated via electrical measurements in the sciatic and vagus nerves as well as in response to sensory stimuli and motor output in the sciatic nerve. The disclosed setup, using direct measures of nerve activity through CAP recordings and muscle force output offers a powerful technique to affect different peripheral nerves with KHFAC and identify frequency-amplitude regions where specific fiber types may be selectively blocked.

While qualitatively similar, the block threshold curves between the sciatic and vagus nerve differed in their quantitative details. The slow component block thresholds were higher while fast component block thresholds were lower for the vagus nerve (FIG. 7) compared to the sciatic nerve (FIG. 6). In addition, block threshold results from the sensory stimuli and motor output study (FIG. 5) were lower compared to slow component block thresholds from the selective block study results (FIG. 6). This is believed to be a result of the differences in fiber recruitment between electrical and sensory stimulation. Supra-maximal electrical pulses activate all the fibers within the nerve while sensory stimulation only activates a small subset of the fibers.

In addition to the trends shown in FIGS. 5, 6, and 7, changes in rectified and integrated area of each component of the CAP suggest that higher resolutions of selectivity may be feasible with KHFAC. FIG. 6 depicts changes in rectified and integrated area of both the fast and slow components of the CAP as a function of frequency and amplitude. Specific frequency and amplitude pairs demonstrate a preference for blocking specific components of the CAP. For example, labels 2, 3, 5, and 6 depict that there are individual changes in rectified and integrated area of either the fast or slow components with increasing amplitude. As the amplitude increased, the selectivity increased (as observed by increasing reduction in individual component areas). All fibers are blocked once the amplitude is above the threshold for both slow and fast fibers.

Prior computational studies suggested a monotonic relationship with frequency for all fiber types, including small diameter unmyelinated fibers. The present disclosure demonstrates the unexpected result that there exists a non-monotonic relationship.

The use of KHFAC to selectively block conduction of specific fiber-types may enhance the efficacy of treatments while removing unnecessary side-effects. In addition, the use of KHFAC to selectively block conduction may enable more controlled investigation of neural circuits underlying a variety of neural pathologies. This technique could selectively target not only somatic (sensory and motor) pathways, but efferent and afferent autonomic neural pathways, including sympathetic and parasympathetic signaling. For example, a KHFAC-enabled reversible vagotomy would offer many advantages over irreversible vagotomy procedures presently used in both scientific and clinical applications.

Numerous characteristics and advantages have been set forth in the foregoing description, together with details of structure and function. While the invention has been disclosed in several forms, it will be apparent to those skilled in the art that many modifications, additions, and deletions, especially in matters of shape, size, and arrangement of parts, can be made therein without departing from the spirit and scope of the invention and its equivalents as set forth in the following claims. Therefore, other modifications or embodiments as may be suggested by the teachings herein are particularly reserved as they fall within the breadth and scope of the claims here appended.

What is claimed is:

1. A method of selectively blocking a portion of a nerve signal, the method comprising:
providing an electrode around a subject's peripheral nerve;
connecting the electrode to a stimulator;
energizing the electrode with a continuous periodic waveform of at least 50 kHz, resulting in the selective block of one of:
a fast portion of a nerve signal having a conduction velocity greater than 2 m/s; and
a slow portion of the nerve signal having a conduction velocity less than 2 m/s;
wherein the other of the fast portion or the slow portion of the nerve signal is allowed to be conducted substantially unimpeded.

2. The method of claim 1, wherein the periodic waveform provided by the stimulator is about 70 kHz.

3. The method of claim 1, wherein the stimulator is a voltage source.

4. The method of claim 1, wherein the stimulator is a current source.

5. The method of claim 1, wherein the electrode is a tripolar cuff-type electrode.

6. The method of claim 1, wherein the peripheral nerve is a sciatic nerve.

7. The method of claim 1, wherein the peripheral nerve is a vagus nerve.

8. The method of claim 1, wherein the selective nerve block is verified by measuring for a muscular contraction.

9. The method of claim 1, wherein the selective nerve block is verified by measuring for a nerve signal propagation using a second electrode.

10. The method of claim 1, wherein the selective nerve block is verified by measuring for a change in a physiological state or biomarker or initiation of a physiological event.

11. The method of claim 1, wherein the periodic waveform provided by the stimulator is adjusted such that a desired indication of the selective nerve block is observed.

12. The method of claim 11, wherein the periodic waveform provided by the stimulator is adjusted such that a desired indication of the conduction of the non-blocked portion of the nerve signal is observed.
